(19) United States
(12) Patent Application Publication
(10) Pub. No.: US 2024/0182385 A1
(43) Pub. Date: Jun. 6, 2024

(54) PROCESS TO PREPARE LOWER OLEFINS

(71) Applicant: TORRGAS TECHNOLOGY B.V., Amsterdam (NL)

(72) Inventor: Michiel CRAMWINCKEL, Voorburg (NL)

(73) Assignee: TORRGAS TECHNOLOGY B.V., Amsterdam (NL)

(21) Appl. No.: 18/556,174

(22) PCT Filed: Apr. 26, 2022
(86) PCT No.: PCT/EP2022/060982

§ 371 Date: Oct. 19, 2023

(30) Foreign Application Priority Data
Apr. 28, 2021 (NL) 2028087

(51) Int. Cl. C07C 11/06; C07C 1/04

(52) U.S. Cl. CPC C07C 11/06; C07C 1/044; C07C 2523/745

(57) ABSTRACT

The invention is directed to a process to prepare ethylene and propylene from a biomass feedstock wherein the process comprises the following steps: (a) a mild gasification of a torrefied biomass feedstock thereby obtaining a char and a gaseous fraction comprising hydrogen, carbon monoxide and a mixture of gaseous organic compounds; (b) a severe gasification of the gaseous fraction in the absence of the char to obtain a substantially tar-free syngas; (c) a Fischer-Tropsch reaction of the substantially tar-free syngas to obtain a first product mixture comprising of methane and C2+ aliphatic hydrocarbons, and (d) a steam cracking reaction of all or part of the C2+ aliphatic hydrocarbons obtained in step (c) to obtain a second product mixture. Methane as isolated from the first and/or the second product mixture may be combusted to generate heat for the endothermal steam cracking reaction in step (d).

The invention is directed to a process to prepare lower olefins like ethylene and propylene.

Steam crackers play a central role in the production of basic chemicals and require a significant amount of energy to break down hydrocarbons into lower olefins and aromatics. Typically, the reaction is conducted at temperatures of about 850° C. A hydrocarbon feedstock and steam is fed to a tubular reactor which is externally heated by burning fossil fuels, like methane based fuels, making this unit operation one of the largest CO₂ emission sources in the existing petrochemical value chain. A popular measure to reduce the CO₂ emissions is by powering the process with electricity. It is stated that by using electricity from renewable sources it is possible to reduce CO₂ emissions by as much as 90%. Recently published examples of steam cracker furnaces based on electricity are described in U.S. Ser. No. 10/351,422, WO20035574, WO20245016, EP3249028, EP3249027 and WO20002326.

A disadvantage of an electrified steam cracker process will still produce considerable volumes of fuels, like methane, LPG and gasoline. These fossil derived fuels will in most cases be used in a combustion process thereby generating fossil derived CO₂. This fossil derived CO₂ is therefore also part of the carbon footprint of an electrified steam cracker operation.

An alternative process to prepare lower olefins is the so-called methanol to olefins process (MTO). The methanol is prepared from coal or natural gas derived syngas. Methanol is then converted to lower olefins. The process is typically applied near natural gas reserves which are far away from large consumer markets like in the Middle East or when coal derived syngas is readily available like in China. A problem of the MTO process is the complexity of the process due to the use of zeolitic catalyst, coke formation and the formation of by-products. Further the MTO process has a considerable carbon footprint.

US2014128486 describes a process which makes lower olefins directly from synthesis gas by first performing a Fischer-Tropsch process. The described process first prepares ethylene, propylene and aliphatic hydrocarbons having 4 or more carbon atoms. The aliphatic hydrocarbons having 4 or more carbons is catalytically converted to ethylene and propylene in the presence of an olefin metathesis catalyst.

The present invention aims to avoid the disadvantages of the present steam cracker processes and the disadvantages of the electrified steam cracker processes as described above.
This is achieved by the following process. Process to prepare ethylene and propylene from a biomass feedstock wherein the process comprises the following steps:
(a) a mild gasification of a torrefied biomass feedstock thereby obtaining a char and a gaseous fraction comprising hydrogen, carbon monoxide and a mixture of gaseous organic compounds;
(b) a severe gasification of the gaseous fraction in the absence of the char to obtain a substantially tar-free syngas;
(c) a Fischer-Tropsch reaction of the substantially tar-free syngas to obtain a first product mixture comprising of methane and C2+ aliphatic hydrocarbons,
(d) a steam cracking reaction of all or part of the C2+ aliphatic hydrocarbons obtained in step (c) to obtain a second product mixture.

Applicant found that with the process according to the invention a process which can convert a biomass feedstock to lower olefins obtained with lower fossil derived CO₂ emissions. The formation of char in step (a) and its use may even result in negative CO₂ emissions. A high yield to lower olefins is achieved by combination of steps (c) and (d). The fuels, like methane, LPG and gasoline which are formed may find use as bio-derived fuels having almost no carbon footprint. A further advantage is that the process may be integrated in an existing steam cracker process wherein step (d) is performed in the furnaces of the existing steam cracker process and wherein the separation train of the existing steam cracker process is used to separate the ethylene and propylene from the first and second product mixtures. The methane which is typically used to heat the furnaces may be the methane formed in steps (c) and/or (d). Because the methane is biomass derived the resulting CO₂ in the flue gas will not negatively influence the carbon footprint of the process. When CO₂ is isolated, suitably as liquid CO₂, in the steam cracker separation train further CO₂ credits may be obtained. A next advantage is that an existing steam cracker process using fossil fuels, like ethane, naphtha or condensates as feedstock and/or recycle streams like waste plastic pyrolysis oils can be gradually transformed to a process using exclusively a biomass feedstock or biomass feedstock and such pyrolysis oils by gradually replacing some of the existing furnace(s) by unit operations performing steps (a)-(c) and maintaining some existing furnaces for performing step (d) while making use of the existing separation train. Further advantages will be described below.

In step (a) a torrefied biomass feedstock is used. The feedstock has been obtained by torrefaction of a starting material comprising lignocellulosic material. Such a process not only increases the heating value per mass biomass by torrefaction but may also remove a substantial amount of water, especially so-called bound-water, from the starting material comprising lignocellulosic material, further also referred to as biomass material. The energy density of the biomass material is increased by decomposing all or part of the hemicelluloses as present in the biomass. An advantage of using a torrefied biomass feed is that the properties of torrefied biomass feeds obtained from different biomass sources may be more uniform than the properties of the original biomass sources. This simplifies the operation of the process according to the invention.

Torrefaction is a well-known process and for example described in WO2012/102617 and in the earlier referred to publication of Prins et al. in Energy and is sometimes referred to as roasting. In such a process the biomass is heated to an elevated temperature, suitably between 260 and 310° C. and more preferably between 250 and 290° C., in the absence of oxygen. Torrefaction conditions are so chosen that hemicelluloses decomposes while keeping the celluloses and lignin intact. These conditions may vary for the type of biomass material used as feed. The temperature and residence time of the torrefaction process is further preferably so chosen that the resulting material has a high content of so-called volatiles, i.e. organic compounds. The solids residence time is suitably at least 5 and preferably at least 10 minutes. The upper residence time will determine the amount of volatiles which remain in the torrefied biomass. Preferably the content of biomass volatiles is between 50 and 80 wt %, more preferably between 60 and 80 wt % and even more preferably between 65 and 75 wt %. The volatile content is measured using DIN 51720-2001-03. Applicants found that the relatively high volatile content in the torrefied biomass is advantageous to achieve a more carbon efficient process from biomass to the char product and the gaseous fraction.

In the torrefaction process the atomic hydrogen over carbon (H/C) ratio and the atomic oxygen over carbon (O/C) ratio of the biomass material is reduced. Preferably the solid torrefied biomass feed has an atomic hydrogen over carbon (H/C) ratio of between 0.7 and 1.3, preferably between 1 and 1.2 and an atomic oxygen over carbon (O/C) ratio of between 0.4 and 0.6. Further the water content will reduce in a torrefaction process. The solid torrefied biomass suitably contains less than 7 wt %, and more preferably less than 4 wt % water, based on the total weight of the solid torrefied biomass.

The biomass material to be torrefied may be any material comprising hemicellulose including virgin biomass and waste biomass. Virgin biomass includes all naturally occurring terrestrial plants such as trees, i.e. wood, bushes and grass. Waste biomass is produced as a low value by-product of various industrial sectors such as the agricultural and forestry sector. Examples of agriculture waste biomass are corn stover, sugarcane bagasse, beet pulp, rice straw, rice hulls, barley straw, corn cobs, wheat straw, canola straw, rice straw, oat straw, oat hulls and corn fibre. A specific example is palm oil waste such as oil palm fronds (OPF), roots and trunks and the by-products obtained at the palm oil mill, such as for example empty fruit bunches (EFB), fruit fibers, kernel shells, palm oil mill effluent and palm kernel cake. Examples of forestry waste biomass are saw mill and paper mill discards. For urban areas, the best potential plant biomass feedstock includes vegetable processing waste and yard waste, for example grass clippings, leaves, tree clippings, and brush. Waste biomass may also be Specified Recovered Fuel (SRF) comprising lignocellulose.

The biomass material to be torrefied may be a mixture originating from different lignocellulosic feedstocks. Furthermore, the biomass feed may comprise fresh lignocellulosic compounds, partially dried lignocellulosic compounds, fully dried lignocellulosic compounds or a combination thereof.

In steps (a) and (b) a considerable amount of the carbon as present in the torrefied biomass feed is converted to gaseous hydrocarbons and eventually to the desired hydrogen and carbon monoxide for use in step (c). The remaining char will comprise ash forming compounds and carbon. By isolating the char and thus also isolating the ash forming as composed within the char from the gaseous reaction products it is possible to subject these gaseous reaction products to the severe gasification temperature conditions of step (b) without the risk that undesired slag will form. The combination of steps (a) and (b) allows to prepare a syngas in a relatively simple manner which syngas is tar free and suited for use in step (c). Step (a) and step (b) may be performed by the process described in WO2020/055254 of applicant.

The torrefied biomass may be used in step (a) as a powder. Preferably the torrefied biomass in step (a) are particles of compressed torrefied biomass. The use of such compressed particles result in that the char is obtained as particles having substantially the same shape as the starting particle.

The particles of a solid torrefied biomass feed may be obtained by pressing the torrefied powder into a shape. The pressing process may be a an extrusion process as described in US2019/0218371, a briquetting process or a pelletising process, for example as performed using a flat die mill or a ring die mill as a pellet mill. Such particles may have any shape, such as cylinders, pillow shape like in briquettes, cubes. The pressing process may be as Preferably the smallest distance from the surface of such a particle to its centre is less than 10 mm. This is advantageous for mass transport within the particle while performing the mild gasification process. For example a suitable particle may be a pellet having the shape of a cylinder suitably having a diameter of between 5 and 12 mm and preferably between 5 and 10 mm. The length of such cylinders may be between 5 and 100 mm, preferably between 40 and 80 mm and more preferably between 40 and 70 mm.

The particles of solid torrefied biomass may have to be transported over long distances and by means of different means of transportation, for example by ocean going ships, river ships and/or trucks. In the loading and transport a significant part of the particle is lost due to dust formation. To avoid the dust formation additives are added to the particles. Preferably the additive is a powder of a plastic, more preferably a powder of a waste plastic. Additionally the waste plastic will enhance the formation of syngas in steps (a) and (b). Thus in this manner not only biomass is converted to lower olefins but also a chemical recycle for waste plastics is provided. The waste plastic preferably comprises polyolefins. The content of waste plastic in the particle is preferably between 1 and 20 wt % and more preferably between 2 and wt % and most preferably below 5 wt %. The powder of a waste plastic may be obtained by milling and more preferably by means of cryogenic milling as for example described in WO2021/048351.

The waste plastic is preferably a mixture of different waste polymers. The mixture suitably comprises two polymers of the following list of polymers consisting of LDPE (Low-density polyethylene), HDPE (High-density polyethylene); PP (Polypropylene); PS (Polystyrene); PVC (Polyvinyl chloride); PET (Polyethylene terephthalate); PUT (Polyurethanes) and PP&A fibres (Polyphthalamide fibres), PVC (polyvinylchloride), polyvinylidene chloride, PU (polyurethane), ABS (acrylonitrile-butadiene-styrene), nylon and fluorinated polymers.

Preferably the waste polymers are substantially only hydrocarbons consisting of only carbon, hydrogen and optionally oxygen. This avoids the formation of nitrogen based combustion gasses and chlorine gasses when PVC is present. Small amounts of these other polymers may be present as contaminants. Preferably the mixture of different waste polymer products at least comprises two polymers of the following list of polymers consisting of LDPE (Low-density polyethylene), HDPE (High-density polyethylene); PP (Polypropylene) and PS (Polystyrene) and PET (Polyethylene terephthalate). Preferably the powder of the waste plastic comprise for more than 50 wt %, more preferably for more than 70 wt %, even more preferably for more than 80 wt % and even more preferably for more than 95 wt % of the listed polymers above. A higher conversion to hydrogen and carbon monoxide may be achieved when these polymers are present.

Other plastics, such as polyvinylchloride, polyvinylidene chloride, polyurethane (PU), acrylonitrile-butadiene-styrene (ABS), nylon and fluorinated polymers are less desirable. The powder of the waste plastic suitably comprises less than 50% by weight, preferably less than 30 wt. %, more preferably less than 20 wt. %, even more preferably less than 10 wt. % of these listed less desirable polymers.

Applicants found that when a particle of compressed torrefied biomass is used a char product, as a char particle, having a high active surface is obtained. The char particle can be used in various applications, such a soil enhancer, activated carbon, filler in engineered plastics, metallurgical coal or can be easily converted in products having favourable end uses.

The mild gasification of step (a) is preferably performed at so-called non-slagging conditions. This avoids the formation of slag and thus no special measures have to be taken for discharge of the slag and/or protection of the process equipment against the slag or molten slag. The latter enables one to use simpler process equipment. These non-slagging conditions are achieved by performing the process at a temperature of between 500 and 800° C. and at a solid residence time of between 10 and 60 minutes. The residence time will be chosen within the claimed range such that the reduction in atomic hydrogen over carbon (H/C) ratio of the solids in the mild gasification process is greater than 50%, preferably greater than 70% and the reduction in atomic oxygen over carbon (O/C) ratio of the solids is greater than 80%. The char particles as obtained preferably have an atomic hydrogen over carbon (H/C) ratio of between 0.02 and 0.1 and an atomic oxygen over carbon (O/C) ratio of between 0.01 and 0.06.

The absolute pressure at which steps (a)-(c) are performed may vary between 90 kPa and 10 MPa and preferably between 90 kPa and 5 MPa.

In the mild gasification process a gaseous fraction comprising hydrogen, carbon monoxide and a mixture of gaseous organic compounds and a solid fraction comprising of char particles is obtained. The gaseous organic compounds may comprise of non-condensed organic compounds. These compounds range from methane to organic compounds having up to 50 carbons and even more. The organic compounds include hydrocarbons and oxygenated hydrocarbons. The fraction of these organic compounds in the gaseous fraction may be greater than wt %. The gaseous fraction may also contain sulphur compounds, such as hydrogen sulphide, sulphinated hydrocarbons and chlorine containing compounds like hydrogen chloride and nitrogen containing compounds like ammonia and hydrogen cyanide. The amount of the latter compounds will depend on the composition of the feed material.

The mild gasification may be performed by contacting the torrefied biomass with an oxygen comprising gas and wherein the amount of oxygen is preferably between 0.1 and 0.3 mass oxygen per mass biomass.

Preferably the mild gasification is performed in the presence of oxygen and steam. The amount of oxygen in such a process also involving steam is suitably between 0.1 and 0.4 kg per kg of the solid biomass feed. The content of oxygen in the combined oxygen steam fraction is suitably between 20 and 40 vol. % O₂ per combined O₂ and H₂O at 300° C.

Step (a) is preferably performed in a continuous process wherein the biomass feed is continuously fed to a reactor and contacted with the oxygen comprising gas. The reactor is preferably an elongated furnace wherein the biomass is continuously transported from a solids inlet at one end of an elongated furnace to a solids outlet at the other end of the elongated furnace. The elongated furnace is preferably a tubular furnace. The means to move the biomass along the length of the reactor may be by means of a rotating wall and/or by rotating means within the furnace. In case of a rotating wall a rotary kiln furnace may be used as for example described in DE19720417 and U.S. Pat. No. 5,769,007. Preferably a tubular elongated reactor is used having rotating means within the furnace. Such rotating means may be an axle positioned axially in the tubular reactor provided with radially extending arms which move the biomass axially when the axle rotates. More preferably such a reactor is further provided with two or more means to supply the oxygen comprising gas, optionally in admixture with steam, along the length of the elongated reactor and between the solids inlet and solids outlet. These inlets for gas are axially spaced apart.

The char as prepared in step (a) is separated from the gaseous fraction before performing step (b). This separation may be performed as a separate step and using any known solids-gas separation technique at high temperature, suitably between 600 and 1000° C., to avoid condensation of the heavy hydrocarbons between performing step (a) and (b). Because the char particles are relatively large no special measures are required to separate the char particles from the gaseous fraction. The particles are suitably separated from the gaseous fraction by means of simple gravitational forces. For example, the particles may be obtained via a discharge at the lower end of a separator while the gaseous fraction is discharged at a higher elevation. Any entrained solids in this gaseous fraction may be separated by means of a cyclone. More preferably use is of filters, like candle filters.

Step (b) is performed by subjecting the gaseous fraction of step (a) to a severe gasification. In step (b) the gaseous organic compounds are converted to hydrogen and carbon monoxide. Because step (b) is performed in the absence of the char formed in step (a) no ash forming compounds will be present in step (b). In this way the formation of molten slag is thus avoided in such a severe gasification process.

In step (b) any methane and higher carbon number hydrocarbons and possible oxygenates as may be present in the gaseous fraction are converted to hydrogen and carbon monoxide thereby obtaining a syngas containing no or almost no tars. The gaseous fraction is subjected to a severe gasification at a temperature of between 1000 and 1600° C. and preferably between 1100 and 1600° C., more preferably between 1200 and 1500° C., and at a residence time of less than 5 seconds, more preferably at a residence time of less than 3 seconds. The residence time is the average gas residence time in the severe gasification reactor. The severe gasification is performed by reaction of oxygen and optionally in the presence of steam, with the organic compounds as present in the gaseous fraction, wherein a sub-stoichiometric amount of oxygen relative to the combustible matter as present in the gaseous fraction is used.

In addition to the gaseous fraction obtained in step (a) also other hydrocarbons may be subjected to the severe gasification in step (b). For example methane obtained in a fermentation process of for example manure. When a high carbon efficiency to lower olefins is desired based on the starting torrefied biomass it is preferred to subject methane, ethane, propane, butane and/or any other higher carbon hydrocarbons as separated from the first and/or second product mixtures to the severe gasification of step (b). A preferred fraction to be co-gasified in step (b) are the high boiling compounds prepared in step (c) which have a too high boiling point to be used as feed in step (d). These additional hydrocarbons may be mixed with the gaseous fraction of step (a) and gasified as a mixture or may be supplied separately to the gasification reactor in which step (b) is performed. For example the gaseous fraction of step (a) may be co-fed to one channel of a multi-annular burner and a liquid feed of high boiling compounds may be fed to another channel of the same multi-annular burner as for example described in EP343735.

A suitable severe gasification process for is for example the Shell Gasification Process as described in the Oil and Gas Journal, Sep. 6, 1971, pp. 85-90. In such a process the gaseous fraction and an oxygen comprising gas is provided to a burner placed at the top of a vertically oriented reactor vessel. Publications describing examples of severe gasification processes are EP291111, WO9722547, WO9639354 and WO9603345.

The oxygen comprising gas used in step (a) and (b) (on a dry basis) comprises preferably at least 90 vol % oxygen, more preferably at least 94 vol % oxygen, wherein nitrogen, carbon dioxide and argon may be present as impurities. Substantially such pure oxygen is preferred, such as prepared by an air separation unit (ASU) or by a water splitter, also referred to as electrolysis. The air compression energy required for the air separation unit is preferably electrical energy and/or rotational energy generated by a steam turbine using steam obtained in step (b) and/or step (c). The total amount of oxygen fed to step (a) and (b) is preferably between 0.1 and 0.6, and more preferably between 0.2 and 0.5 mass oxygen per mass torrefied biomass as fed to step (a). This may be higher when significant amounts of hydrocarbons are co-gasified in step (b) as described in this specification.

The syngas as obtained in the severe gasification will have an elevated temperature. Suitably the syngas is cooled in a boiler generating steam. The steam or super heated steam as obtained may be used to generate electricity for use in the process of this invention or for other uses. Alternatively or in combination the syngas mixture may be contacted with a carbonaceous compound to chemically quench the syngas mixture. By directly contacting the syngas mixture with a carbonaceous compound an endothermic reaction will take place thereby reducing the temperature of the resulting gas mixture. For this reason, the term chemical quenching is used. The quenching compounds may be the earlier referred to compounds and fractions which may be co-gasified in step (b).

The syngas obtained in step (b) is suitably conditioned to be used in step (c). This may involve cooling, pressurising and gas treating to remove impurities like H₂S, COS, NH₃, HCN and alkali and halides and adaptation of the hydrogen to carbon monoxide molar ratio. The required purity and H₂/CO molar ratio may be different for different Fischer-Tropsch catalyst systems used in step (c).

An additional source of syngas is suitably provided by reforming carbon dioxide and methane or by a reserve water shift reaction (RWGS). The carbon dioxide is suitably carbon dioxide separated from the first product mixture and/or second product mixture. The methane is suitably methane separated from the first product mixture and/or second product mixture. Alternative or additional methane sources may be used to convert the carbon dioxide. Such alternative or additional methane may be derived from natural gas, refinery off gas and/or methane formed in a steam cracker process. A preferred methane comprising gas is biogas obtained in an anaerobic digesting process, for example an anaerobic digesting process of livestock manure. The use of biogas, directly and/or via certificates, is preferred when the olefins require to be made from a biomass source. By obtaining biogas certificates, also referred to as ‘green as certificates’, matching a volume of natural as from the natural gas grid consumed in the reforming process will allow to claim that the lower olefins like ethylene and propylene, are biobased. By using the substantial volume of carbon dioxide which may be prepared in step (c) to make syngas by reaction with methane it is possible to keep the carbon dioxide within the process. It serves as a chemical flywheel. In the reserve water shift reaction (RWGS) carbon dioxide reacts with hydrogen to syngas.

This CO₂ conversion step is advantageous when in step (c) relatively large amounts of methane and carbon dioxide are formed, especially when Fischer-Tropsch catalysts are used which are selective for lower olefins. By reacting the methane and carbon dioxide to syngas a higher carbon efficiency is achieved. The reforming reaction may be performed in the presence of a suitable catalyst as described in EP1180495. The required heat may be provided by combustion of methane, preferably methane separated from the first product mixture and/or second product mixture. Preferably the required heat is provided by combustion of hydrogen. The hydrogen may be isolated from the syngas, especially when the hydrogen to carbon monoxide ratio of the produced syngas is too high for the chosen Fischer-Tropsch catalyst.

The reforming catalyst is preferably present in tubes which are externally heated. The furnace and convection section of such a dry-reforming reactor may suitably be a retrofitted steam cracker furnace which has become obsolete because a substantial amount of the lower olefins are now prepared by the process of this invention.

The required heat may also be provided by electrically heating the tubes in which the reforming catalyst is present. Even more preferred the reforming is performed as a plasma dry reforming process. In such a process a plasma of carbon dioxide is prepared using for example electromagnetic waves generated by electricity. The plasma is contacted with methane to form syngas as for example described in WO20223789. US2015246337. US2016121296.

In step (c) a Fischer-Tropsch reaction of the substantially tar-free syngas is performed. The Fischer-Tropsch reaction may be performed using a cobalt, iron, ruthenium and nickel catalyst. Preferably iron or cobalt based catalysts are used. Iron based catalysts are preferred because less removal of impurities are required. Further iron based catalysts have the advantage that the olefin to paraffin ratio in the first product mixture may be higher as compared to some cobalt based catalyst systems. This is advantageous because this would result in more C2-C4 olefin formation in step (c) thereby increasing the C2-C4 olefin selectivity of the entire process. By a Fischer-Tropsch reaction which is selective for ethylene and propylene is meant that of the hydrocarbons produced more than 20 wt %, preferably more than 30 wt % and even more preferably more than 40 wt % of the hydrocarbons produced in the Fischer-Tropsch reaction are ethylene, propylene and olefins having 4 carbons. Although a high selectivity to olefins is desired the upper limit for this ethylene and propylene selectivity may in practice be a value smaller than 60 wt %.

The carbon number distribution in the first product mixture of methane and the C2+ aliphatic hydrocarbons obtained in step (c) varies for a chosen Fischer-Tropsch catalyst, reactor type and reaction conditions. It is know that the products formed in a state of thermodynamic equilibrium follow Anderson-Schulz-Flory's general polymerization distribution at carbon numbers greater than about three. Typically in gas to liquids processes starting from methane, e.g. natural gas, Fischer-Tropsch catalysts are favoured which have a high selectivity to the higher carbon number compounds and a resulting low selectivity for methane. Such catalyst systems may be used. The significant fraction of high carbon paraffins formed by these catalyst systems may not be readily used as feedstock in step (d). Especially when step (d) is performed in existing steam cracking furnaces which are designed for lower boiling fossil feedstocks, for example designed for a fossil derived naphtha feedstock and/or designed for a gas condensate feedstock. These high boiling fractions, suitably after being separated from the Fischer-Tropsch synthesis products obtained in step (c), may be subjected to a hydrocracking or thermal cracking process to obtain lower boiling compounds which may be used in step (d). Suitably, for example when such additional unit operations are less preferred, Fischer-Tropsch catalyst system and reaction conditions which are more selective for methane and C2-C4 aliphatic hydrocarbons are used. Even more preferred are Fischer-Tropsch catalyst system and reaction conditions which are also more selective for C2-C4 olefins and a C5+ fraction comprising evaporative olefins and paraffins. By evaporative olefins and paraffins is here meant compounds boiling substantially in the boiling range for which an existing fossil based steam cracker furnace is designed for. Formed methane in step (c) may be used to generate the required heat to perform the endothermal steam cracking reaction in step (d). This biomass derived methane may also be converted to products instead of using it as fuel. This may be by reacting the methane with the formed carbon dioxide to prepare syngas as described elsewhere in this description.

The Fischer-Tropsch catalyst may comprise iron, cobalt, ruthenium and nickel. Preferably the catalyst comprises iron, cobalt or ruthenium. Ruthenium is the most active catalytic metal but also not readily available. For this reason cobalt and iron is preferred. The metal may be present on a support and the catalyst may further comprise a promoter. Examples of cobalt comprising Fischer-Tropsch An example of a suitable Fischer-Tropsch catalyst comprising cobalt is the cobalt, manganese and zinc comprising catalyst as described in EP2422876, the cobalt, manganese and one element of lanthanum and phosphorus as described in US2014/0128486. Iron comprising catalysts are preferred because they typically require a lower hydrogen to carbon monoxide molar ratio which aligns better with the lower ratio obtained in step (b) of the current process. Further iron based catalyst are less sensitive for impurities in the syngas which simplifies the gas treating of the syngas before it is used in step (c). Further the product slate of an iron catalysed step (c) is more directed to the desired methane and lower carbon number aliphatic hydrocarbons. Examples of iron comprising catalysts are described in EP2490989 illustrating a catalyst comprising iron oxide particles on an alpha-alumina support iron-oxide, in WO2014/210090 illustrating a catalyst comprising iron, potassium on an yttria support. Other examples of suitable Fischer-Tropsch catalysts are described in US2017/0173565, US2016/0121311, EP2694457, US20180134967, CN101219384, CN109534939, CN107362802, CN104801304, WO14210090, CN103071543 and WO2016/185334.

The reaction conditions in step (c) may depend on the type of catalyst and reactor. The hydrogen to carbon monoxide molar ratio in the feed to step (c) may range from 0.5 to 4. For a cobalt Fischer-Tropsch catalyst the favoured H₂/CO molar ratio may be at the upper end of this range while for many iron based Fischer-Tropsch catalysts the favoured H₂/CO molar ratio may be at the lower end of this range. Cobalt based systems directed to selectively make lower olefins are known to use a syngas having lower favoured H₂/CO molar ratio. The reaction temperature in step (c) may be between 150 and 350° C., a space velocity of about 400-5000 h-1 and a pressure of between atmospheric to about 5 MPa.

The Fischer-Tropsch reactor may be a slurry bubble column reactor, a multitubular trickle bed reactor, a circulating fluidized bed reactor and a fixed fluidized bed reactor. A multitubular reactor is preferred because it can be applied in modules of a certain size or sizes. Thus when an existing steam cracker process is gradually converted from a fossil based process to a biomass based process according to the invention it is preferred that the Fischer-Tropsch reactor capacity can be gradually increased. By using multitubular reactors such a gradual increase is possible by simply adding reactor tubes as part of added reactors.

In step (c) a first product mixture will be obtained comprising of water, unconverted syngas, carbon dioxide, methane and gaseous C2+ aliphatic hydrocarbons and liquid aliphatic hydrocarbons. The first product mixture is suitably separated into a FT gaseous fraction comprising methane, water, unconverted syngas, carbon dioxide and C2-C4 aliphatic hydrocarbons and a liquid C5+ fraction of aliphatic hydrocarbons. Next to these aliphatic hydrocarbons also oxygenates may be present.

Preferably in step (c) a Fischer-Tropsch reaction is performed which is selective for ethylene and propylene. These processes may result in a first product comprising of between 10 and 50 wt % carbon dioxide. For such processes it is preferred to isolate all or part of the carbon dioxide as liquid carbon dioxide. Liquid carbon dioxide can be easily stored and transported to other downstream processes. Carbon dioxide as obtained from a biomass source can be used as a sustainable feedstock to make sustainable non-fossil derived products, such as for example diethyl carbonate as described in WO21037516 and propylene carbonate as described in US2020/0399239.

Preferably carbon dioxide is isolated and reacted with methane and/or hydrogen to react to carbon monoxide and especially to prepare a syngas. The syngas may be used to prepare for example methanol or middle distillates such as gas oil and/or kerosene. Preferably the syngas is used as an additional feedstock in step (c). This syngas may have a high hydrogen to carbon monoxide molar ratio making it also suitable as a feedstock to prepare bio-methanol or to prepare middle distillates, like gas oil and kerosene. Such middle distillates may be prepared by using the syngas as feed to a Fischer-Tropsch process selective to make a paraffin wax product and subsequently hydrocracking the wax to the desired middle distillates. The carbon dioxide may be reacted with hydrogen in the earlier referred to reserve water gas shift (RWGS) process to syngas. The source of hydrogen may be the hydrogen as isolated in a separation train which is used to separate the olefin products from the first and second product mixtures. In addition or as an alternative source hydrogen may be used which is obtained by water splitting using sustainable electrical energy source, like wind and solar. An example of such a RWGS process is described in WO21244980.

The C5+ fraction of aliphatic hydrocarbons may be steam cracked in step (d) as such. Because the fraction may contain high carbon number hydrocarbons special measures may have to be taken to fully evaporate this feed and avoid coking issues. Because the fraction comprises almost only of aliphatic hydrocarbons and no aromatics a low coke make is expected. When the process is performed in a steam cracker process which is designed for propane, naphtha or gas oil feeds it may be advantageous to separate the C5+ fraction in a FT-distillate and a FT residue. The FT distillate may suitably boil substantially below a temperature of between 250 and 370° C. and the FT residue will substantially boil above a temperature of between 250 and 370° C. The FT distillate is preferably steam cracked in step (d). The FT distillate is a very good feedstock to make lower olefins in a high yield due to the fact that it is highly paraffins and contains no aromatic compounds. If the FT distillate contains olefins it may be advantageous to subject the FT distillate to a hydrogenation step to saturate the olefins before performing a steam cracking in step along (d). This avoids rapid coking in the steam cracking step.

The amount of FT residue as part of the first product mixture is not high when a step (c) is performed aiming at a high selectivity to lower olefins and methane. Low amounts of separated FT residue may be used as quench oil in the steam cracking process wherein the aliphatic hydrocarbons may thermally crack to lower boiling compounds. The FT residue may also be used as co-feed in the severe gasification of step (b) to prepare syngas. When the higher boiling FT residue has a higher volume, for example when step (c) is performed at a ASF-alpha value (Anderson-Schulz-Flory chain growth factor derived from the C20 compounds and the C40 compounds of the Fischer-Tropsch product stream) of at least 0.925, it may be interesting to subject this fraction to a hydroconversion process to prepare middle distillates like gas oil and kerosene. The obtained fraction boiling below the kerosene boiling range, which is less suited to be used as transportation fuel, may advantageously be steam cracked in step (d).

An examples of such a hydroconversion process is a platinum supported on a silica-alumina carrier catalysed hydroconversion process as described in for example EP0532117. Hydrogen required for such processes may be isolated from the first and/or second product mixtures and/or may be prepared from the syngas obtained in step (b) by performing a water gas shift reaction. Methane formed in such a hydroconversion is typically considered as a disadvantage. In the present process this methane may be advantageously combusted to generate heat for the endothermal steam cracking reaction in step (d) without increasing the carbon footprint.

When an existing steam cracker is converted to a biomass based process of this invention it may be advantageous to first add the first product mixture of step (c) directly to the separation train of the existing steam cracker process. Preferably the first product mixture is added to the oil quench. If the first product mixture is a pressurised product mixture it may be preferred to add this product mixture to a suited pressure level in a compressor train of the existing separation train or directly to a cold box of the existing separation train, optionally after separating CO₂ and/or a C5+ fraction from the first product mixture. The main part of the steam cracker furnaces may then still be fed by a fossil based feed while part of the methane combusted to generate heat for the endothermal steam cracking reaction of this fossil fuel is biomass derived. When more biomass capacity is added it will become advantageous to add the above described separation units to isolate the C5+ fraction or the FT distillate and use these directly as feed to step (d) to more fully take advantage of the present invention.

When gradually replacing fossil fuel based feeds by biobased feeds more lower olefins will be produced from a biomass feed. By using mass balance calculations it is possible to assign part of the lower olefins produced as biobased or green olefins making use of International Sustainability and Carbon Certification (ISCC Plus) system.

When the Fischer-Tropsch process step (c) is performed at elevated pressures it may be preferred to directly supply the pressurised gaseous products, preferably after separating the C5+ fraction, to a cold box of a separation train of an existing steam cracker process. Before or after passing the cold box CO₂ is suitably separated from the pressurised gaseous products. In such a CO2 recovery system a CO₂ rich stream, preferably as a liquid, is separated from the hydrocarbons of the gaseous products.

It is preferred to remove at least 80 vol percent, preferably at least 90 vol percent, more preferably at least 95 vol percent and at most 99.5 vol percent of the CO₂ as is present in the gaseous products.

On an industrial scale there are chiefly two categories of absorbent solvents, depending on the mechanism to absorb the CO₂: chemical solvents and physical solvents. Each solvent has its own advantages and disadvantages as to features as loading capacity, kinetics, regenerability, selectivity, stability, corrosivity, heat/cooling requirements etc.

Chemical solvents which have proved to be industrially useful are primary, secondary and/or tertiary amines derived alkanolamines. The most frequently used amines are derived from ethanolamine, especially monoethanol amine (MEA), diethanolamine (DEA), triethanolamine (TEA), diisopropanolamine (DIPA) and methyldiethanolamine (MDEA).

Physical solvents which have proved to be industrially suitable are cyclo-tetramethylenesulfone and its derivatives, aliphatic acid amides, N-methylpyrrolidone, N-alkylated pyrrolidones and the corresponding piperidones, methanol, ethanol and mixtures of dialkylethers of polyethylene glycols.

A well-known commercial process uses an aqueous mixture of a chemical solvent, especially DIPA and/or MDEA, and a physical solvent, especially cyclotetramethylene-sulfone. Such systems show good absorption capacity and good selectivity against moderate investment costs and operational costs. They perform very well at high pressures, especially between 20 and 90 bara.

The physical absorption process is preferred and is well known to the man skilled in the art. Reference can be made to e.g. Perry, Chemical Engineerings' Handbook, Chapter 14, Gas Absorption. The liquid absorbent in the physical absorption process is suitably methanol, ethanol, acetone, dimethyl ether, methyl i-propyl ether, polyethylene glycol or xylene, preferably methanol. This process is based on carbon dioxide and hydrogen sulfide being highly soluble under pressure in the methanol, and then being readily releasable from solution when the pressure is reduced as further discussed below. This high pressure system is preferred due to its efficiency, although other removal systems such as using amines are known. The physical absorption process is suitably carried out at low temperatures, preferably between −60 degrees centigrade and 0 degrees centigrade, preferably between −30 and −10 degrees centigrade

The physical absorption process is carried out by contacting the gaseous products in a counter-current upward flow with the liquid absorbent. The absorption process is preferably carried out in a continuous mode, in which the liquid absorbent is regenerated. This regeneration process is well known to the man skilled in the art. The loaded liquid absorbent is suitably regenerated by pressure release (e.g. a flashing operation) and/or temperature increase (e.g. a distillation process). The regeneration is suitably carried out in two or more steps, preferably 3-10 steps, especially a combination of one or more flashing steps and a distillation step.

The regeneration of solvent from the process is also known in the art. Preferably, the present invention involves one integrated solvent regeneration tower. Further process conditions are for example described in DE2610982 and DE4336790.

In step (d) an aliphatic comprising feed is used which comprises of all or parts of the first and second product mixtures after separating at least ethylene and propylene. This feed is steam cracked to obtain the second product mixture. The steam cracking reaction may be performed under the steam cracking conditions know to the skilled person. Preferably a mixture of hydrocarbons and steam is fed to a radiant coil as present in a radiant section of a steam cracker furnace at about 0.2 to about 1 kg steam per kg hydrocarbon. Steam cracking conditions can include one or more of (i) a temperature of the mixture from 760 to 880° C. (ii) a pressure within the radiant coil from 1 to 5 bar, and/or (iii) a cracking residence time from 0.1 to 2 seconds. The effluent of a radiant coil can have a temperature from 760 to 1100° C., preferably to about 900° C.

Step (d) may be performed in a conventional steam cracker furnace wherein methane is combusted to generate heat for the endothermal steam cracking reaction. The methane may be natural gas and/or the methane as separated from the first and/or the second product mixture. Instead of methane also hydrogen or mixtures comprising of methane and hydrogen may be combusted to generate heat for the endothermal steam cracking reaction. The hydrogen may be non-converted hydrogen of step (c), hydrogen as formed in step (d) and/or hydrogen as isolated from the, suitably water gas shifted, syngas as prepared in step (b). The CO₂ as obtained in such a water gas shift reaction may be reacted to syngas with the methane in a reforming process step as described above.

Preferably step (d) is performed in steam cracking furnaces of an existing fossil based steam cracker process. This allows one to combust methane or the hydrogen based fuels as described above to generate heat for the endothermal steam cracking reaction and also make use the existing separation train of such a process to separate the first and second product mixtures and especially the gaseous fraction of the first product mixture together with the second product mixture. This avoids having to electrify the steam cracker furnaces. This may be preferred in situations where the required high power consumption require huge adaptations to an existing power grid. In this way the electricity as generated by renewable energy may be used to avoid reduction of CO₂ emissions elsewhere.

A steam cracker separation train typically comprises of a steam boiler and an oil quench to rapidly cool the second product mixture, a quench oil recovery unit, a pyrolysis gasoline recovery unit, an output for a pyrolysis gasoline product, a compressor unit, consisting of a series of compressors, a sour gas washing unit, a cold box and a distillation train having outputs for an ethylene product, a propylene product, ethane, propane, and lights comprising methane, hydrogen and carbon monoxide. Further outlets for a C4-C5 fraction, a C5-C9 fraction, a C4 and a C5 fraction may be present. From the C4 fraction olefins like butadiene may be recovered. From the C5-C9 fraction, also referred to as pygas, aromatics like benzene, xylene and toluene may be isolated. The ethane as isolated is suitably steam cracked in step (d). Typically a specially designed ethane furnace is present to optimally convert ethane to predominantly ethylene. Propane, the C4-C5 fraction, the C4 fraction and/or the C5 fraction may be steam cracked in step (d). Alternatively propane may be marketed as a biomass based LPG product. The obtained pyrolysis gasoline may be marketed as a biomass based gasoline. CO₂ may be isolated in the sour gas washing unit and advantageously used as biomass derived or green CO₂ as a feedstock in other processes. Examples of steam cracker separation trains are described in U.S. Pat. Nos. 5,372,009, 5,361,589 and 8,552,245.

To avoid a build-up of quench oil it may be advantageous to co-feed part of the quench oil to the severe gasification of step (b).

The lights may be combusted as a mixture to generate heat for the endothermal steam cracking reaction in step (d) or may be separated wherein hydrogen and/or carbon monoxide are separated from methane. The hydrogen and/or carbon monoxide may be recycled to step (c). The hydrogen may be used the above mentioned hydroconversion process to make middle distillates, in the hydrogenation step to saturate olefins in a olefin containing FT distillate and/or to saturate any olefins as present in a pyrolysis gasoline obtainable from the second product mixture.

The invented process may also be performed in steam cracker reactors wherein the required heat for the endothermal steam cracking reaction is provided from an electrical source. This is advantageous because no CO₂ will be emitted to the environment as part of a flue gas. As a consequence a large amount of methane is available for other uses. One advantageous use is to reform this methane with the obtained carbon dioxide to prepare additional syngas for use in step (c). This may allow for converting CO₂ as separated from the first product mixture and optionally also from the second product mixture.

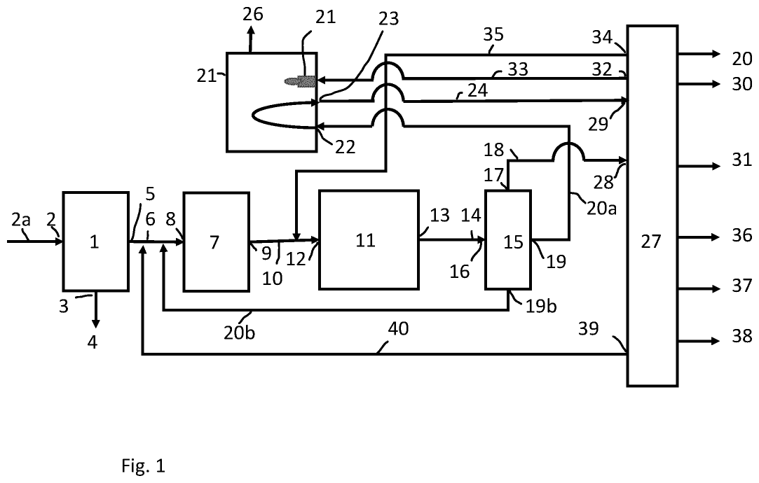

The invention will be illustrated making use of the following FIGS. 1-4. The FIG. 1 shows a process configuration to prepare ethylene and propylene suited to perform the process of this invention. The process configuration comprises the following units (i), (ii), (iii), (iv) and (v):
(i) one or more mild gasification reactor units (1) having an inlet (2) for a torrefied biomass feedstock (2a); an outlet (3) for a char product (4) and an outlet (5) for a gaseous fraction (6);
(ii) one or more severe gasification reactor units (7) having an inlet (8) fluidly connected to the outlet (5) for the gaseous fraction of the one or more mild gasification reactor units (1) and an outlet (9) for a substantially tar-free syngas (10); one or more Fischer-Tropsch reactor units (11) having an inlet (12) fluidly connected to the outlet (9) for a substantially tar-free syngas and further comprising of a heterogeneous Fischer-Tropsch catalyst and an outlet (13) for a first product mixture (14);
(iii) a separation unit (15) having an inlet (16) for the first product mixture (14) and an outlet (17) for a FT gaseous fraction (18) and an outlet (19) for a FT C5+ fraction or alternatively a FT C5+ distillate (20a) and an outlet (19b) for a FT residue (20b) as shown;
(iv) one or more furnaces (21) for performing a steam cracking reaction having an inlet (22) fluidly connected to the outlet (19) for the FT distillate, optionally via a hydrogenation step, and wherein the furnaces (21) have an outlet (23) for a second product mixture (24) and wherein the furnaces (21) are provided with burners (25) for combustion of methane generating a flue gas which is emitted to the environment via outlet (26). From these flue gasses CO₂ may be removed by for example amine absorption processes. The obtained CO₂ may have a carbon credit value because it is biomass derived;
(v) a separation train (27) having an inlets (28,29) fluidly connected to the outlet (17) for a FT gaseous fraction and fluidly connected to the outlet (23) for a second product mixture (24). The separation train (27) has multiple outlets for various products of which the outlet (30) for a ethylene product, an outlet (31) for a propylene product, an outlet (32) for methane (33) and an outlet (20) for carbon dioxide. The outlet (32) for methane (33) may be fluidly connected to the burners (25) of the furnaces (21) and an outlet (34) for hydrogen and carbon monoxide (35) may be fluidly connected to the inlet (12) of a Fischer-Tropsch reactor unit (11). Further an outlet (39) is shown for a quench oil purge (40) fluidly connected to the inlet (8) of the one or more severe gasification reactor units (7). Further product outlets are drawn for butadiene (36), pyrolysis gasoline (37) and for BTX aromatics (38) as illustrative examples of the multitude of products which may be isolated in a separation train of a steam cracker process.

Between the one or more severe gasification reactor units (7) and the one or more Fischer-Tropsch reactor units (11) gas treating units may be present to remove any catalyst poisons. Depending on the Fischer-Tropsch catalyst between 0 and almost all of the sulphur compounds and/or nitrogen compounds, like for example HCN, have to be removed from the syngas.

In a large scale process configuration more than one mild gasification reactor units (1) operating in parallel may be fluidly connected to more than one severe gasification reactor units (7) operating in parallel. These severe gasification reactor units (7) may in turn be fluidly connected to one Fischer-Tropsch reactor unit (11), for example a slurry phase reactor, or to more than one Fischer-Tropsch reactor units (11), for example multitubular reactors, operating in parallel. The one or more Fischer-Tropsch reactor units (11) may in turn be fluidly connected to one separation unit (15). The separation unit (15) may be fluidly connected to more than one furnaces (21), for example existing furnaces of a steam cracking process which have previously been used to run on fossil derived feeds. Next to these furnaces (21) dedicated furnaces for recycle streams may be present, such as a furnace for recycle ethane. Such a furnace may also be an existing furnace designed for converting recycle ethane. The number of parallel operating units as described above may be the same or different. Thus for example two mild gasification reactor units (1) may be fluidly connected to one severe gasification reactor units (7).

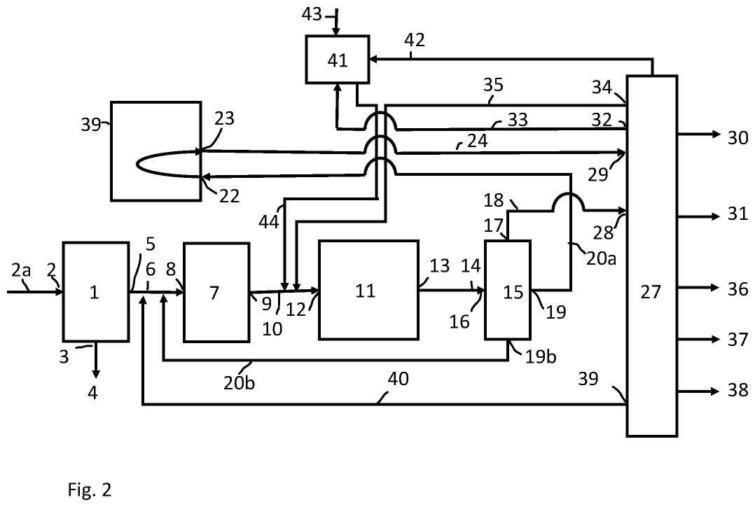

FIG. 2 shows a process like the process of FIG. 1 except in that the heat required to perform the endothermal cracking reactions in the steam cracker furnace (39) is generated by electricity. Thus no flue gas outlet (26) is present. The methane (33) is now fed to a plasma reformer (41). Further CO₂ (42) as separated in the separation train (27) and external CO₂ (43) is fed to plasma reformer (41) to prepare syngas (44). When the selectivity to carbon dioxide is high in the Fischer-Tropsch step it may even be desired to add additional methane, preferably biomethane, to this process via stream (43) instead of additional carbon dioxide. This additional syngas (44) is fed to the inlet (12) of the Fischer-Tropsch reactor unit (11). The hydrogen to carbon monoxide ratio of the additional syngas (44) may be too high for performing the Fischer-Tropsch reaction. Hydrogen is then preferably separated from the syngas, for example by means of a membrane separation. If enough hydrogen is generated in this manner it may even be desirable to perform the dry methane reforming as in (41) in a conventional furnace, instead of a plasma reformer, using the hydrogen as fuel for externally heating the reactor tubes. The hydrogen may be used the above mentioned hydroconversion process to make middle distillates, in the hydrogenation step to saturate olefins in a olefin containing FT distillate and/or to saturate any olefins as present in a pyrolysis gasoline obtainable from the second product mixture (24).

When the dry methane reforming is performed in a conventional furnace as for example described in EP1180495 it may be advantageous to retrofit an existing steam cracker furnace to become a furnace suited to perform the dry methane reforming step. Because more olefins will be directly produced as part of the first gaseous product part of the existing steam cracker furnaces of an existing steam cracker process will become obsolete. One or more of such obsolete furnaces may be retrofitted to furnaces to perform the dry-methane reforming.

This process has a high carbon efficiency because of this reuse of methane and CO₂. Processes combining the furnaces (21) of FIG. 1 and the plasma (41) of FIG. 2 or conventional dry methane reformer are of course also possible. Processes combining the furnaces (21) of FIG. 1 and the furnaces of FIG. 2 and optionally the plasma (41) of FIG. 2 or conventional dry methane reformer are of course also possible.

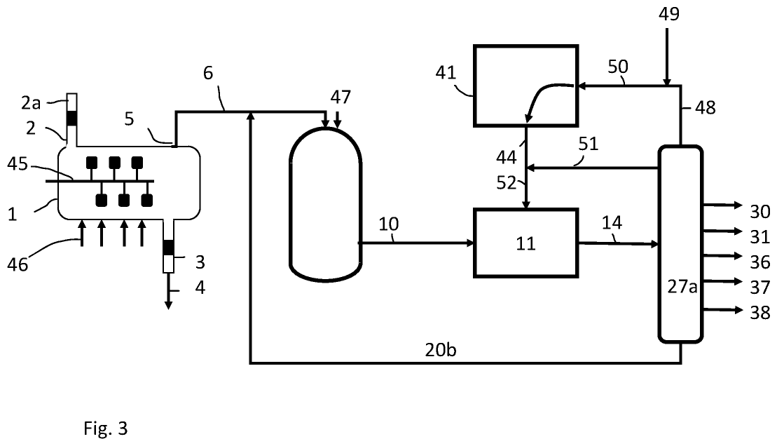

FIG. 3 describes a process to prepare ethylene and propylene from a biomass feedstock. The same reference signs relate to the same elements of FIGS. 1 and 2. In addition a rotating axle with arms is shown to move the biomass along an elongated reactor (1). Along the length of the elongated reactor (1) a mixture of oxygen and steam is supplied at more than one axially spaced apart supply points (46). To the severe gasification reactor unit (7) oxygen (47) is supplied. The gaseous fraction is subjected to a severe gasification by reaction with a sub stoichiometric amount of oxygen of the in the absence of the char to obtain a substantially tar-free syngas which is used in the Fischer-Tropsch step (FT). In the FT a first product mixture 4) comprising of more than 20 wt % carbon dioxide is obtained and further comprising methane, ethane, ethylene, propane, propylene, C4 olefins and paraffins and C5+ aliphatic hydrocarbons. From the separation train (27a) methane and carbon dioxide (48) are isolated and mixed with methane (49) to prepare the optimal feed composition (50) for plasma reformer (41). If the hydrogen to carbon monoxide mol ratio of the syngas made in plasma reformer (41) is too high or low adjustments may be made. Typical adjustments are for example the water gas shift reaction to increase the hydrogen content or a membrane separation to remove part of the hydrogen. In addition or instead carbon monoxide (51) as isolated in the separation train (27) may be added to adapt this ratio. In this way the optimal syngas composition (52) for performing the Fischer-Tropsch reaction in the Fischer-Tropsch reactor unit (11) may be obtained. From the first product mixture (14) C5+ aliphatic hydrocarbons (20b) are separated and recycled to the severe gasification reactor unit (7).

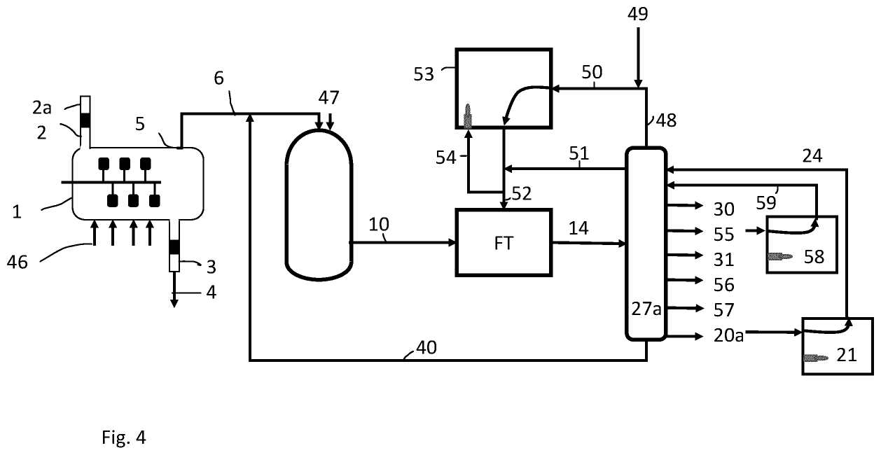

FIG. 4 shows a variant of the process of FIG. 3 wherein the process is integrated with an existing steam cracker process. A FT residue (40) is isolated from the first product mixture and recycled to the severe gasification reactor unit (7) and a FT distillate is steam cracked in step (d) in steam cracker furnace (21). The dry reforming is now performed in a retrofitted steam cracker furnace (53) being fueled by a hydrogen comprising fuel (54) resulting in a flue gas (53a) having lowered CO₂ emissions. The hydrogen is separated from the syngas obtained in the dry reforming. Further the ethane (55) is steam cracked to lower olefins, preferably making use of an existing ethane steam cracking furnace (58). The product mixture (59) obtained in ethane steam cracking furnace (58) is separated in separation train (27a). Next a naphtha fraction isolated as a low boiling fraction from the FT product mixture, optionally after being subjected to a hydrotreatment to remove olefins, is steam cracked to lower olefins, preferably in an existing naphtha steam cracker furnace. In this way part of the existing furnaces of a steam cracker and its downstream separation train (27a) may be advantageously used. The hydrocarbons boiling above the naphtha fraction, the high boiling fraction, referred to as tars in the Figure, are gasified to syngas in the severe gasification.

The invention is therefore also directed to a process to prepare ethylene and propylene from a biomass feedstock wherein the process comprises the following steps:
(aa) a gasification of a biomass feedstock thereby obtaining a substantially tar-free syngas;
(bb) a Fischer-Tropsch reaction of the substantially tar-free syngas to obtain a product mixture comprising of more than 10 wt % carbon dioxide, preferably between 10 and 50 wt %, more preferably between 20 and 40 wt %, and further comprising ethylene, propylene, methane and C5+ aliphatic hydrocarbons,
(cc) isolating carbon dioxide, preferably as a liquid product, ethylene and propylene from the product mixture.

Preferably a step (dd) is performed wherein the carbon dioxide is reformed with methane to prepare a syngas and/or converted with hydrogen by a reserve water shift reaction (RWGS) to prepare a syngas and using the syngas, optionally after adapting the hydrogen to carbon monoxide mol ratio, in step (cc).

The biomass may be any source of biomass, for example woody biomass or fibrous biomass. Preferably the biomass is a torrefied biomass as described in this application and more preferably the gasification is (aa1) a mild gasification of a torrefied biomass feedstock thereby obtaining a char and a gaseous fraction comprising hydrogen, carbon monoxide and a mixture of gaseous organic compounds and (aa2) a severe gasification of the gaseous fraction in the absence of the char to obtain a substantially tar-free syngas as described in this application.

Preferably the C5+ aliphatic hydrocarbons obtained in step (bb) are gasified to syngas in the severe gasification (bb) as described earlier in this description. The C5+ aliphatic hydrocarbons obtained in step (bb) are suitably separated into a low boiling fraction, the FT distillate, which is steam cracked to obtain ethylene and propylene and a high boiling fraction, the FT residue, which is gasified to syngas in the severe gasification (aa2). The ethane is suitably isolated from the product mixture and which ethane is steam cracked to obtain ethylene and propylene. Preferably the steam cracking of the ethane is performed in an existing steam cracking furnace of an existing steam cracker process. Further preferred embodiments for performing step (aa) are described above for step (a). Further preferred embodiments for performing step (bb) are described above for step (c). Further preferred embodiments for performing step (dd) are described above when describing the reforming step and the reserve water shift reaction (RWGS).

The isolated carbon dioxide is either used as feedstock to prepare chemicals as described above and/or used to prepare syngas as described above.

1-27. (canceled)

28. A process to prepare ethylene and propylene from a biomass feedstock wherein the process comprises the following steps:
(a) a mild gasification of a torrefied biomass feedstock thereby obtaining a char and a gaseous fraction comprising hydrogen, carbon monoxide and a mixture of gaseous organic compounds;
(b) a severe gasification of the gaseous fraction in the absence of the char to obtain a substantially tar-free syngas;
(c) a Fischer-Tropsch reaction of the substantially tar-free syngas to obtain a first product mixture comprising of methane and C2+ aliphatic hydrocarbons,
(d) a steam cracking reaction of all or part of the C2+ aliphatic hydrocarbons obtained in step (c) to obtain a second product mixture.

29. The process according to claim 28, wherein in step (c) a Fischer-Tropsch reaction is performed which is selective for ethylene and propylene and wherein the first product mixture is obtained comprising of between 10 and 50 wt % carbon dioxide, wherein all or part of the carbon dioxide is isolated to obtain isolated carbon dioxide.

30. The process according to claim 29, wherein the isolated carbon dioxide is reacted with a methane feedstock in a reforming step to a syngas and using the syngas as an additional feedstock in step (c).

31. The process according to claim 30, wherein the reforming step is a plasma dry reforming process or a dry-reforming process.

32. The process according to claim 30, wherein the methane feedstock comprises methane obtained in an anaerobic digesting process directly and/or via certificates.

33. The process according to claim 29, wherein the first product mixture is separated into a FT gaseous fraction and a C5+ fraction and wherein the aliphatic comprising feed of step (d) is comprised of this C5+ fraction.

34. The process according to claim 33, wherein the C5+ fraction is separated in a FT distillate boiling substantially below 370° C. and a FT residue boiling substantially above 370° C. and wherein the aliphatic comprising feed step (d) is comprised of this the FT distillate.

35. The process according to claim 28, wherein ethane is separated from the first and from the second product mixture and wherein the ethane is subjected to a steam cracking reaction in a step (e) to obtain a third product mixture.

36. The process according to claim 35, wherein ethylene, propylene are isolated from the FT gaseous fraction, from the second product mixture and from the third product mixture in the same separation train.

37. The process according to claim 36, wherein step (d) and step (e) are performed in steam cracker furnaces of an existing steam cracker process and wherein at least part of the separation train is of the existing steam cracker process and wherein the existing steam cracker process is designed and build to convert a naphtha feedstock which naphtha feedstock is replaced by the biomass feedstock and the optional methane feedstock.

38. The process according to claim 28, wherein in step (a) the torrefied biomass feedstock in step (a) is least one of:
i) particles of a solid torrefied biomass having a content of biomass volatiles of between 60 and 80 wt %; and
ii) particles comprising between 1 and 20 wt % of a waste plastic.

39. The process according to claim 28, further comprising at least one of:
i) performing step (a) at a temperature of between 500 and 800° C. and at a solid residence time of between 10 and 60 minutes and step (b) at a temperature of between 1000 and 1600 C;
ii) performing step (a) in the presence of oxygen and steam.

40. The process according to claim 28, further comprising performing step (c) in a multitube reactor using a Fischer-Tropsch catalyst comprising iron.

41. A process system to prepare ethylene and propylene from a biomass feedstock wherein the process configuration comprises the following units:
(i) a mild gasification reactor unit in series with;
(ii) a severe gasification reactor unit to produce a substantially tar-free syngas;
(iii) a Fischer-Tropsch reactor unit for converting the substantially tar-free syngas to a first product mixture wherein the Fischer-Tropsch reactor unit comprises of a heterogeneous Fischer-Tropsch catalyst and an outlet for a first product mixture;
(iv) a separation unit for separating the first product mixture into at least a FT gaseous fraction and a higher boiling FT C5+ fraction;
(v) one or more furnaces for performing a steam cracking reaction wherein at least one furnace is suited to convert the entire FT C5+ fraction or a distillate of the FT C5+ fraction to a second product mixture;
(vi) a separation train for isolating ethylene and propylene from the first product mixture and from the second product mixture.

42. A process to prepare ethylene and propylene from a biomass feedstock wherein the process comprises the following steps:
(aa) a gasification of a biomass feedstock thereby obtaining a substantially tar-free syngas;
(bb) a Fischer-Tropsch reaction of the substantially tar-free syngas to obtain a product mixture comprising of more than 20 wt % carbon dioxide, preferably between 10 and 50 wt %, more preferably between 20 and 40 wt %, and further comprising ethylene, propylene, methane and C5+ aliphatic hydrocarbons, and
(cc) isolating carbon dioxide, ethylene and propylene from the product mixture.

43. The process according to claim 42, wherein the process further comprises at least one of:
i) isolating the carbon dioxide as a liquid product;
ii) reforming the carbon dioxide with methane to prepare a syngas; and
iii) converting the carbon dioxide with hydrogen by a reverse water shift reaction (RWGS) to prepare a syngas; and
iv) using the syngas in step (cc).

44. The process according to claim 42, wherein step (aa) comprises a mild gasification of a torrefied biomass feedstock thereby obtaining a char and a gaseous fraction comprising hydrogen, carbon monoxide and a mixture of gaseous organic compounds and a severe gasification of the gaseous fraction in the absence of the char to obtain a substantially tar-free syngas.

45. The process according to claim 44, wherein the torrefied biomass feedstock are pellets of the torrefied biomass feedstock.

46. The process according to claim 42, wherein the C5+ aliphatic hydrocarbons are separated into a low boiling fraction which is steam cracked to obtain ethylene and propylene and a high boiling fraction which is gasified to syngas in the severe gasification.

47. The process according to claim 42, wherein ethane is isolated from the product mixture and which ethane is steam cracked to obtain ethylene and propylene.
